SEQUENCE LISTING (20 sequences)

SEQ ID NO: 1 - protein, 185 aa, Bacillus subtilis
ASTDYWQNWTDGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 2 - protein, 185 aa, synthetic construct
  REGION 1..185: note = S22E mutein of B. subtilis xylanase
ASTDYWQNWTDGGGIVNAVNGEGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 3 - protein, 185 aa, synthetic construct
  REGION 1..185: note = D11Y_S22E mutein of B. subtilis xylanase
ASTDYWQNWTYGGGIVNAVNGEGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 4 - protein, 185 aa, synthetic construct
  REGION 1..185: note = D11F_S22E mutein of B. subtilis xylanase
ASTDYWQNWTFGGGIVNAVNGEGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 5 - protein, 185 aa, synthetic construct
  REGION 1..185: note = D11Y_S22E_R122D mutein of B. subtilis xylanase
ASTDYWQNWTYGGGIVNAVNGEGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDDTTFTQYWSVRQSKRPTGSNATITFSNHVNAWKSHGMNLGSNWAYQVMATEGYQSSGSSNVTVW

SEQ ID NO: 6 - protein, 185 aa, synthetic construct
  REGION 1..185: note = D11F_S22E_R122D mutein of B. subtilis xylanase
ASTDYWQNWTFGGGIVNAVNGEGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDDTTFTQYWSVRQSKRPTGSNATITFSNHVNAWKSHGMNLGSNWAYQVMATEGYQSSGSSNVTVW

SEQ ID NO: 7 - protein, 28 aa, Bacillus subtilis
MFKFKKKFLVGLSAALMSISLFSATASA

SEQ ID NO: 8 - protein, 213 aa, Bacillus subtilis
MFKFKKKFLVGLSAALMSISLFSATASAASTDYWQNWTDGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 9 - protein, 213 aa, synthetic construct
  REGION 1..213: note = S22E mutein of B. subtilis xylanase w/signal peptide
MFKFKKKFLVGLSAALMSISLFSATASAASTDYWQNWTDGGGIVNAVNGEGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 10 - protein, 213 aa, synthetic construct
  REGION 1..213: note = D11Y_S22E mutein of B. subtilis xylanase w/ signal peptide
MFKFKKKFLVGLSAALMSISLFSATASAASTDYWQNWTYGGGIVNAVNGEGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 11 - protein, 213 aa, synthetic construct
  REGION 1..213: note = D11F_S22E mutein of B. subtilis xylanase w/ signal peptide
MFKFKKKFLVGLSAALMSISLFSATASAASTDYWQNWTFGGGIVNAVNGEGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDRTTFTQYWSVRQSKRPTGSNATITFSNHVNAWKSHGMNLGSNWAYQVMATEGYQSSGSSNVTVW

SEQ ID NO: 12 - protein, 213 aa, synthetic construct; identical to SEQ ID NO: 10
  REGION 1..213: note = D11Y_S22E_R122D mutein of B. subtilis xylanase w/ signal peptide

SEQ ID NO: 13 - protein, 213 aa, synthetic construct; identical to SEQ ID NO: 11
  REGION 1..213: note = D11F_S22E_R122D mutein of B. subtilis xylanase w/ signal peptide

SEQ ID NO: 14 - protein, 185 aa, synthetic construct
  REGION 1..185: note = D11F_R122D mutein of B. subtilis xylanase (WO 01/66711 A1)
ASTDYWQNWTFGGGIVNAVNGSGGNYSVNWSNTGNFVVGKGWTTGSPFRTINYNAGVWAPNGNGYLTLYGWTRSPLIEYYVVDSWGTYRPTGTYKGTVKSDGGTYDIYTTTRYNAPSIDGDDTTFTQYWSVRQSKRPTGSNATITFSNHVNAWKSHGMNLGSNWAYQVMATEGYQSSGSSNVTVW

SEQ ID NO: 15 - DNA, 642 bp, Bacillus subtilis
  source 1..642: mol_type = genomic DNA
atgtttaagtttaaaaagaatttcttagttggattatcggcagctttaatgagtattagcttgttttcggcaaccgcctctgcagctagcacagactactggcaaaattggactgatgggggcggtatagtaaacgctgtcaatgggtctggcgggaattacagtgttaattggtctaataccggaaattttgttgttggtaaaggttggactacaggttcgccatttaggacgataaactataatgccggagtttgggcgccgaatggcaatggatatttaactttatatggttggacgagatcacctctcatagaatattatgtagtggattcatggggtacttatagacctactggaacgtataaaggtactgtaaaaagtgatgggggtacatatgacatatatacaactacacgttataacgcaccttccattgatggcgatcgcactacttttacgcagtactggagtgttcgccagtcgaagagaccaaccggaagcaacgctacaatcactttcagcaatcatgtgaacgcatggaagagccatggaatgaatctgggcagtaattgggcttaccaagtcatggcgacagaaggatatcaaagtagtggaagttctaacgtaacagtgtggtaa

SEQ ID NO: 16 - DNA, 642 bp, synthetic construct
  misc_feature 1..642: note = Xylanase S22E encoding
  source 1..642: mol_type = other DNA
atgtttaagtttaaaaagaatttcttagttggattatcggcagctttaatgagtattagcttgttttcggcaaccgcctctgcagctagcacagactactggcaaaattggactgatgggggcggtatagtaaacgctgtcaatggggaaggcgggaattacagtgttaattggtctaataccggaaattttgttgttggtaaaggttggactacaggttcgccatttaggacgataaactataatgccggagtttgggcgccgaatggcaatggatatttaactttatatggttggacgagatcacctctcatagaatattatgtagtggattcatggggtacttatagacctactggaacgtataaaggtactgtaaaaagtgatgggggtacatatgacatatatacaactacacgttataacgcaccttccattgatggcgatcgcactacttttacgcagtactggagtgttcgccagtcgaagagaccaaccggaagcaacgctacaatcactttcagcaatcatgtgaacgcatggaagagccatggaatgaatctgggcagtaattgggcttaccaagtcatggcgacagaaggatatcaaagtagtggaagttctaacgtaacagtgtggtaa

SEQ ID NO: 17 - DNA, 642 bp, synthetic construct
  misc_feature 1..642: note = Xylanase D11Y_S22E encoding
  source 1..642: mol_type = other DNA
atgtttaagtttaaaaagaatttcttagttggattatcggcagctttaatgagtattagcttgttttcggcaaccgcctctgcagctagcacagactactggcaaaattggacttatgggggcggtatagtaaacgctgtcaatggggaaggcgggaattacagtgttaattggtctaataccggaaattttgttgttggtaaaggttggactacaggttcgccatttaggacgataaactataatgccggagtttgggcgccgaatggcaatggatatttaactttatatggttggacgagatcacctctcatagaatattatgtagtggattcatggggtacttatagacctactggaacgtataaaggtactgtaaaaagtgatgggggtacatatgacatatatacaactacacgttataacgcaccttccattgatggcgatcgcactacttttacgcagtactggagtgttcgccagtcgaagagaccaaccggaagcaacgctacaatcactttcagcaatcatgtgaacgcatggaagagccatggaatgaatctgggcagtaattgggcttaccaagtcatggcgacagaaggatatcaaagtagtggaagttctaacgtaacagtgtggtaa

SEQ ID NO: 18 - DNA, 642 bp, synthetic construct
  misc_feature 1..642: note = Xylanase D11F_S22E encoding
  source 1..642: mol_type = other DNA
atgtttaagtttaaaaagaatttcttagttggattatcggcagctttaatgagtattagcttgttttcggcaaccgcctctgcagctagcacagactactggcaaaattggacttttgggggcggtatagtaaacgctgtcaatggggaaggcgggaattacagtgttaattggtctaataccggaaattttgttgttggtaaaggttggactacaggttcgccatttaggacgataaactataatgccggagtttgggcgccgaatggcaatggatatttaactttatatggttggacgagatcacctctcatagaatattatgtagtggattcatggggtacttatagacctactggaacgtataaaggtactgtaaaaagtgatgggggtacatatgacatatatacaactacacgttataacgcaccttccattgatggcgatcgcactacttttacgcagtactggagtgttcgccagtcgaagagaccaaccggaagcaacgctacaatcactttcagcaatcatgtgaacgcatggaagagccatggaatgaatctgggcagtaattgggcttaccaagtcatggcgacagaaggatatcaaagtagtggaagttctaacgtaacagtgtggtaa

SEQ ID NO: 19 - DNA, 642 bp, synthetic construct
  misc_feature 1..642: note = Xylanase D11Y_S22E_R122D encoding
  source 1..642: mol_type = other DNA
atgtttaagtttaaaaagaatttcttagttggattatcggcagctttaatgagtattagcttgttttcggcaaccgcctctgcagctagcacagactactggcaaaattggacttatgggggcggtatagtaaacgctgtcaatggggaaggcgggaattacagtgttaattggtctaataccggaaattttgttgttggtaaaggttggactacaggttcgccatttaggacgataaactataatgccggagtttgggcgccgaatggcaatggatatttaactttatatggttggacgagatcacctctcatagaatattatgtagtggattcatggggtacttatagacctactggaacgtataaaggtactgtaaaaagtgatgggggtacatatgacatatatacaactacacgttataacgcaccttccattgatggcgatgacactacttttacgcagtactggagtgttcgccagtcgaagagaccaaccggaagcaacgctacaatcactttcagcaatcatgtgaacgcatggaagagccatggaatgaatctgggcagtaattgggcttaccaagtcatggcgacagaaggatatcaaagtagtggaagttctaacgtaacagtgtggtaa

SEQ ID NO: 20 - DNA, 642 bp, synthetic construct
  misc_feature 1..642: note = Xylanase D11F_S22E_R122D encoding
  source 1..642: mol_type = other DNA
atgtttaagtttaaaaagaatttcttagttggattatcggcagctttaatgagtattagcttgttttcggcaaccgcctctgcagctagcacagactactggcaaaattggacttttgggggcggtatagtaaacgctgtcaatggggaaggcgggaattacagtgttaattggtctaataccggaaattttgttgttggtaaaggttggactacaggttcgccatttaggacgataaactataatgccggagtttgggcgccgaatggcaatggatatttaactttatatggttggacgagatcacctctcatagaatattatgtagtggattcatggggtacttatagacctactggaacgtataaaggtactgtaaaaagtgatgggggtacatatgacatatatacaactacacgttataacgcaccttccattgatggcgatgacactacttttacgcagtactggagtgttcgccagtcgaagagaccaaccggaagcaacgctacaatcactttcagcaatcatgtgaacgcatggaagagccatggaatgaatctgggcagtaattgggcttaccaagtcatggcgacagaaggatatcaaagtagtggaagttctaacgtaacagtgtggtaa

(19) United States
(12) Patent Application Publication
(10) Pub. No.: US 2025/0011746 A1
(43) Pub. Date: Jan. 9, 2025

(54) IMPROVED XYLANASES

(71) Applicant: AB Enzymes GmbH, Darmstadt (DE)

(72) Inventors: Tina PLOSS, Darmstadt (DE); Felix FISCHER, Darmstadt (DE); Daniela LINNERT, Darmstadt (DE); Yasar KAYA, Darmstadt (DE); Andreas HOLKENBRINK, Darmstadt (DE); Carsten HINRICHS, Darmstadt (DE); Evelyn WALLIS, Darmstadt (DE)

(73) Assignee: AB Enzymes GmbH, Darmstadt (DE)

(21) Appl. No.: 18/708,474

(22) PCT Filed: Nov. 8, 2022
(86) PCT No.: PCT/EP2022/081119

§ 371 Date: May 8, 2024

(30) Foreign Application Priority Data
Nov. 9, 2021 (EP) 21207285.4

(51) Int. Cl. C12N 9/24; A21D 8/04; C12N 15/75

(52) U.S. Cl. CPC C12N 9/2402; A21D 8/042; C12N 15/75; C12Y 302/01008

(57) ABSTRACT

The present invention relates to a xylanase polypeptide comprising (i) an amino acid sequence at least 70% identical to SEQ ID NO: 1, (ii) an amino acid exchange at the position corresponding to amino acid D11 in SEQ ID NO: 1; and (iii) an amino acid exchange at the position corresponding to amino acid S22 in SEQ ID NO: 1. The present invention further relates to polynucleotides, host cells, methods, compositions, and uses related thereto.

The present invention relates to a xylanase polypeptide comprising (i) an amino acid sequence at least 70% identical to SEQ ID NO:1, (ii) an amino acid exchange at the position corresponding to amino acid D11 in SEQ ID NO:1; and (iii) an amino acid exchange at the position corresponding to amino acid S22 in SEQ ID NO:1. The present invention further relates to polynucleotides, host cells, methods, compositions, and uses related thereto.

For many years, endo-β-1,4-xylanases (EC 3.2.1.8) have been used for the modification of complex carbohydrates derived from plant cell wall material. Differences between the functionalities of different xylanases have been suggested to be due to differences in xylanase specificity and thereby their preference for water-unextractable arabinoxylan (WU-AX) or water-extractable arabinoxylan (WE-AX) substrates.

In some applications (e.g. bakery), it is desirable to produce high molecular weight (HMW) soluble polymers from the WU-AX fraction. Such polymers have been correlated to a volume increase in bread making (Courtin et al. (1999) J Agric Food Chem. 47(5): 180). In baking applications, glycoside hydrolase family 11 xylanases are particularly of interest. These xylanases can modify WU-AX, resulting in increased dough liquid viscosity in a wheat flour dough, and thereby are very effective in applications where a reduction in viscosity is desired.

Improvement of xylanase properties by introducing amino acid exchanges has been reported, e.g. Ruller et al. (2014) Protein Engineering, Design & Selection 27(8):255 described development of an alkali-tolerant and thermophilic enzyme, while Alptoni et al. (2016), Int J Biol Macromol 87:522 suggested amino acid exchanges for thermostability improvement of a B. subtilis xylanase. Alptoni et al. (loc. cit.) describes an increased specific activity of S22E B. subtilis xylanase variant at temperatures above 40° C. compared to the wt molecule.

Endogenous xylanase inhibitors can affect the activity of xylanases also in wheat flour systems, which is undesirable e.g. in bakery applications. Xylanase variants having reduced sensitivity to xylanase inhibitors and hence altered functionality, are therefore desirable. To that end, WO 01/066711 A1 described Bacillus subtilis xylanase A variants comprising a mutation selected from D11Y, D11F, D11K, D11F/R122D, and D11F/G34D having reduced sensitivity to a xylanase inhibitor; WO 2010/072225 A1 reported amino acid modifications in positions 12 and/or 13.

Nonetheless, there is still a need in the art for improved xylanases and means of production and uses thereof. This problem is addressed by the xylanase polypeptides, methods, products, compositions, kits, and uses with the features of the independent claims. Advantageous embodiments which might be realized in an isolated fashion or in any arbitrary combinations are listed in the dependent claims.

In accordance, the present invention relates to a xylanase polypeptide comprising
(i) an amino acid sequence at least 70% identical to SEQ ID NO:1,
(ii) an amino acid exchange at the position corresponding to amino acid D11 in SEQ ID NO:1; and
(iii) an amino acid exchange at the position corresponding to amino acid S22 in SEQ ID NO:1.

In general, terms used herein are to be given their ordinary and customary meaning to a person of ordinary skill in the art and, unless indicated otherwise, are not to be limited to a special or customized meaning. As used in the following, the terms “have”, “comprise” or “include” or any arbitrary grammatical variations thereof are used in a non-exclusive way. Thus, these terms may both refer to a situation in which, besides the feature introduced by these terms, no further features are present in the entity described in this context and to a situation in which one or more further features are present. As an example, the expressions “A has B”, “A comprises B” and “A includes B” may both refer to a situation in which, besides B, no other element is present in A (i.e. a situation in which A solely and exclusively consists of B) and to a situation in which, besides B, one or more further elements are present in entity A, such as element C, elements C and D or even further elements. Also, as is understood by the skilled person, the expressions “comprising a” and “comprising an” preferably refer to “comprising one or more”, i.e. are equivalent to “comprising at least one”. In accordance, expressions relating to one item of a plurality, unless otherwise indicated, preferably relate to at least one such item, more preferably a plurality thereof; thus, e.g. providing “a cell” relates to providing at least one cell, preferably to providing a multitude of cells.

Further, as used in the following, the terms “preferably”, “more preferably”, “most preferably”, “particularly”, “more particularly”, “specifically”, “more specifically” or similar terms are used in conjunction with optional features, without restricting further possibilities. Thus, features introduced by these terms are optional features and are not intended to restrict the scope of the claims in any way. The invention may, as the skilled person will recognize, be performed by using alternative features. Similarly, features introduced by “in an embodiment” or similar expressions are intended to be optional features, without any restriction regarding further embodiments of the invention, without any restrictions regarding the scope of the invention and without any restriction regarding the possibility of combining the features introduced in such way with other optional or non-optional features of the invention.

The methods specified herein below, preferably, are in vitro methods, preferably are baking applications. The method steps may, in principle, be performed in any arbitrary sequence deemed suitable by the skilled person, but preferably are performed in the indicated sequence; also, one or more, preferably all, of said steps may be assisted or performed by automated equipment. Moreover, the methods may comprise steps in addition to those explicitly mentioned above.

As used herein, the term “standard conditions”, if not otherwise noted, relates to IUPAC standard ambient temperature and pressure (SATP) conditions, i.e. preferably, a temperature of 25° C. and an absolute pressure of 100 kPa; also preferably, standard conditions include a pH of 7. Moreover, if not otherwise indicated, the term “about” relates to the indicated value with the commonly accepted technical precision in the relevant field, preferably relates to the indicated value ±20%, more preferably ±10%, most preferably ±5%. Further, the term “essentially” indicates that deviations having influence on the indicated result or use are absent, i.e. potential deviations do not cause the indicated result to deviate by more than ±20%, more preferably ±10%, most preferably ±5%. Thus, “consisting essentially of” means including the components specified but excluding other components except for materials present as impurities, unavoidable materials present as a result of processes used to provide the components, and components added for a purpose other than achieving the technical effect of the invention. For example, a composition defined using the phrase “consisting essentially of” encompasses any known acceptable additive, excipient, diluent, carrier, and the like. Preferably, a composition consisting essentially of a set of components will comprise less than 5% by weight, more preferably less than 3% by weight, even more preferably less than 1% by weight, most preferably less than 0.1% by weight of non-specified component(s).

The degree of identity (e.g. expressed as “% identity”) between two biological sequences, preferably DNA, RNA, or amino acid sequences, can be determined by algorithms well known in the art. Preferably, the degree of identity is determined by comparing two optimally aligned sequences over a comparison window, where the fragment of sequence in the comparison window may comprise additions or deletions (e.g., gaps or overhangs) as compared to the sequence it is compared to for optimal alignment. The percentage is calculated by determining, preferably over the whole length of the polynucleotide or polypeptide, the number of positions at which the identical residue occurs in both sequences to yield the number of matched positions, dividing the number of matched positions by the total number of positions in the window of comparison and multiplying the result by 100 to yield the percentage of sequence identity. Optimal alignment of sequences for comparison may be conducted by the local homology algorithm of Smith and Waterman (1981), by the homology alignment algorithm of Needleman and Wunsch (1970), by the search for similarity method of Pearson and Lipman (1988), by computerized implementations of these algorithms (GAP, BESTFIT, BLAST, PASTA, and TFASTA in the Wisconsin Genetics Software Package, Genetics Computer Group (GCG), 575 Science Dr., Madison, WI), or by visual inspection. Given that two sequences have been identified for comparison, GAP and BESTFIT are preferably employed to determine their optimal alignment and, thus, the degree of identity. Preferably, the default values of 5.00 for gap weight and 0.30 for gap weight length are used. In the context of biological sequences referred to herein, the term “essentially identical” indicates a % identity value of at least 80%, preferably at least 90%, more preferably at least 98%, most preferably at least 99%. As will be understood, the term essentially identical includes 100% identity. The aforesaid applies to the term “essentially complementary” mutatis mutandis. As will be understood, the expression “at least x % identity” includes all values starting from x % identity, higher than x % identity, as well as 100% identity.

The term “position corresponding” in a first biological sequence, preferably amino acid sequence, to a specific position in a second biological sequence is understood by the skilled person to relate to the position being equivalent to the specific position in the context of the biological sequences. In accordance, a position corresponding in a first biological sequence to a specific position in a second biological sequence preferably is identified by aligning said first and second biological sequences, preferably as specified herein above, and identifying the position in the first biological sequence which is aligned to lie at a position juxtaposed to the specific position in the second biological sequence. As the skilled person understands, further biological sequences may additionally be aligned, e.g. in order to improve alignment quality.

The term “fragment” of a biological macromolecule, preferably of a polynucleotide or polypeptide, is used herein in a wide sense relating to any sub-part, preferably subdomain, of the respective biological macromolecule comprising the indicated sequence, structure and/or function. Thus, the term includes sub-parts generated by actual fragmentation of a biological macromolecule, but also sub-parts derived from the respective biological macromolecule in an abstract manner, e.g. in silico.

The term “polypeptide”, as used herein, refers to a molecule consisting of several, typically at least 30 amino acids that are covalently linked to each other by peptide bonds. Molecules consisting of less than 30 amino acids covalently linked by peptide bonds are usually considered to be “peptides”. Preferably, the polypeptide comprises of from 50 to 1000, more preferably of from 75 to 1000, still more preferably of from 100 to 500, most preferably of from 110 to 400 amino acids. The polypeptide may be comprised in a fusion polypeptide and/or a polypeptide complex.

The term “xylanase polypeptide”, as used herein, relates to a polypeptide having the structural properties, preferably an amino acid sequence, as specified and having xylanase activity. The xylanase polypeptide comprises an amino acid sequence at least 70% identical to SEQ ID NO:1. Preferably, the xylanase polypeptide comprises an amino acid sequence at least 80%, more preferably at least 90%, still more preferably at least 95%, even more preferably at least 97%, even more preferably at least 98%, most preferably at least 99%, identical to SEQ ID NO:1. In view of the description herein, the xylanase polypeptide may also comprise an amino acid sequence at least 80%, more preferably at least 90%, still more preferably at least 95%, even more preferably at least 97%, even more preferably at least 98%, most preferably at least 99%, identical to SEQ ID NO:2, 8, and/or 9. Preferably, the xylanase polypeptide comprises, more preferably consists of, the amino acid sequence of SEQ ID NO:3 or 4 or a sequence at least 90%, preferably at least 95%, more preferably at least 98%, most preferably at least 99% identical thereto. More preferably, the xylanase polypeptide comprises, more preferably consists of, the amino acid sequence of SEQ ID NO:3 or 4, more preferably 3. Also preferably, the xylanase polypeptide comprises, more preferably consists of, the amino acid sequence of SEQ ID NO:5 or 6 or a sequence at least 90%, preferably at least 95%, more preferably at least 98%, most preferably at least 99% identical thereto. More preferably, the xylanase polypeptide comprises, more preferably consists of, the amino acid sequence of SEQ ID NO:5 or 6, more preferably 5. Also preferably, the xylanase polypeptide comprises, more preferably consists of, the amino acid sequence of SEQ ID NO:10 or 11 or a sequence at least 90%, preferably at least 95%, more preferably at least 98%, most preferably at least 99% identical thereto. More preferably, the xylanase polypeptide comprises, more preferably consists of, the amino acid sequence of SEQ ID NO:10 or 11, more preferably 10. Also preferably, the xylanase polypeptide comprises, more preferably consists of, the amino acid sequence of SEQ ID NO:12 or 13 or a sequence at least 90%, preferably at least 95%, more preferably at least 98%, most preferably at least 99% identical thereto. More preferably, the xylanase polypeptide comprises, more preferably consists of, the amino acid sequence of SEQ ID NO:12 or 13, more preferably 12.

The xylanase polypeptide further comprises at least two amino acid exchanges compared to SEQ ID NO:1, as specified herein. As the skilled person understands, an “amino acid exchange” in an amino acid sequence relates to a replacement of an amino acid by a non-identical amino acid. An amino acid exchange may be a conservative exchange, i.e. an exchange of an amino acid for a non-identical amino acid of the same functional group of amino acids. In a conservative amino acid exchange, an amino acid with a hydrophobic side chain (“hydrophobic amino acid”, A, V, L, I, M, F, Y, or W) is exchanged for a non-identical hydrophobic amino acid; an amino acid with a negatively charged side chain (D or E), is exchanged for a non-identical amino acid with a negatively charged side chain; an amino acid with an uncharged polar side chain (S, T, N, or Q) is exchanged for an amino acid with an uncharged polar side chain; an amino acid with a positively charged side chain (R, H, or K) is exchanged for a non-identical amino acid with a positively charged side chain. Preferably, the amino acid exchange as referred to herein is a non-conservative exchange, i.e. an amino acid is exchanged for a non-identical amino acid not having the same or a similar functional group and/or having structurally dissimilar side chain. Thus, preferably, an amino acid is exchanged for an amino acid from a non-identical functional group as specified herein above.

The xylanase polypeptide comprises an amino acid exchange at the position corresponding to amino acid D11 in SEQ ID NO:1. Preferably, the D11 exchange is a non-conservative amino acid exchange, more preferably is a D11Y, a D11F, a D11K, a D11N, a D11K, a D11S, or a D11W exchange, even more preferably is a D11Y, a D11F, or a D11K exchange, most preferably is a D11Y or a D11F exchange, in particular a D11Y exchange.

The xylanase polypeptide further comprises an amino acid exchange at the position corresponding to amino acid S22 in SEQ ID NO:1. Preferably, the S22 exchange is a non-conservative amino acid exchange, more preferably an S22E, an S22D, an S22N, an S22Q, an S22A, an S22V, an S22L, or an S22I exchange, even more preferably an S22E or an S22D exchange, most preferably an S22E exchange.

Optionally, the xylanase polypeptide further comprises an amino acid exchange at the position corresponding to amino acid R122 in SEQ ID NO:1. Preferably, the R122 exchange is a non-conservative amino acid exchange, more preferably is an R122D, an R122N, an R122E, an R122Y, an R122F, an R122K, or an R122A exchange, still more preferably is an R122D or R122N exchange.

Optionally, the xylanase polypeptide further comprises an amino acid exchange at the position corresponding to amino acid G34 in SEQ ID NO:1. Preferably, the G34 exchange is a non-conservative amino acid exchange, more preferably is a G34D exchange. Optionally, the xylanase polypeptide further comprises an amino acid exchange at the position corresponding to amino acid G12 in SEQ ID NO:1. Preferably, the G12 exchange is a G12F exchange. Optionally, the xylanase polypeptide further comprises an amino acid exchange at the position corresponding to amino acid G13 in SEQ ID NO:1. Preferably, the G13 exchange is a G13Y exchange. Optionally, the xylanase polypeptide comprises one or more amino acid exchanges selected from the group consisting of G12F, G13Y, I15Y, G34K, I77V, I77M, I77Y, I77L, I77S, V81I, V82I, K99Y, T104W, THOA, Y113D, Y113A, N114F, N114D, N114Y, I118V, R122F, R122D, K154R, N159D, S162E, S162D, 164F, Y166F, Q175L, Q175K, Q175E, Q175Y, and S179Y.

Optionally, the xylanase further comprises a signal peptide, preferably as specified herein above. More preferably, said signal peptide comprises, preferably consists of, the sequence of SEQ ID NO:7.

As specified herein above, the xylanase polypeptide has xylanase activity, i.e., has detectable xylanase activity, preferably endo-β-1,4-xylanase activity (EC 3.2.1.8). Preferably, said xylanase activity is determined in the assay as specified in Example 1. Preferably, measurement of xylanase activity is performed as described in Example 1 or according to WO 01/66711 A1 in the absence of xylanase inhibitor; thus, preferably, xylanase activity as specified herein in Example 1. The aforesaid xylanase activity preferably is essentially not inhibited by a wheat endogenous xylanase inhibitor, i.e. is not inhibited by more than 20%, more preferably 10%, still more preferably 5%, by a wheat endogenous xylanase inhibitor. More preferably, the xylanase polypeptide has a xylanase activity which is essentially not inhibited by a wheat endogenous xylanase inhibitor at a concentration of 10 xylanase inhibitor units/mL; preferably, measurement of xylanase inhibition is performed according to WO 01/66711 A1, Example 4. Preferably, the xylanase polypeptide is producible in a prokaryotic expression system, preferably as specified herein below, at an at least 1.3-fold, preferably 1.4-fold, more preferably 1.5-fold, even more preferably 1.7-fold, most preferably 2-fold, volume yield compared to a xylanase polypeptide consisting of the amino acid sequence of SEQ ID NO:1 or 14 produced under essentially the same conditions.

Advantageously, it was found in the work underlying the present invention that the xylanase polypeptides of the present invention provide for improved dough and baking properties when used in dough preparation, e.g. decreased dough stickiness and/or increased baking volume. Moreover, it was found that the xylanase polypeptides of the invention can be produced at a higher volume yield, thus reducing production cost. Thus, surprisingly it was found that exchange of amino acid S22 to E increases the xylanase polypeptide volume yield compared to a xylanase polypeptide without said amino acid exchange. Furthermore, it was surprisingly found in the work underlying the present invention that S22E xylanase variants provide for improved dough and baking properties when used in dough preparation, e.g. decreased dough stickiness and/or increased baking volume.

The definitions made above apply mutatis mutandis to the following. Additional definitions and explanations made further below also apply for all embodiments described in this specification mutatis mutandis.

The present invention also relates to a polynucleotide comprising a nucleic acid sequence encoding a xylanase polypeptide according to the present invention.

The term “polynucleotide” is known to the skilled person. As used herein, the term includes nucleic acid molecules comprising or consisting of a nucleic acid sequence or nucleic acid sequences as specified herein. The polynucleotide of the present invention shall be provided, preferably, either as an isolated polynucleotide (i.e. isolated from its natural context) or in genetically modified form. The polynucleotide, preferably, is DNA, including cDNA, or is RNA. The term encompasses single as well as double stranded polynucleotides. Preferably, the polynucleotide is a chimeric molecule, i.e., preferably, comprises at least one nucleic acid sequence, preferably of at least 20 bp, more preferably at least 100 bp, heterologous to the residual nucleic acid sequence(s) or being an artificial nucleic acid sequence. Moreover, preferably, comprised are also chemically modified polynucleotides including naturally occurring modified polynucleotides such as glycosylated or methylated polynucleotides or artificial modified ones such as biotinylated polynucleotides.

As used herein, the term polynucleotide, preferably, includes variants of the specifically indicated polynucleotides. More preferably, the term polynucleotide relates to the specific polynucleotides indicated. It is to be understood, however, that a polypeptide having a specific amino acid sequence may be encoded by a variety of polynucleotides, due to the degeneration of the genetic code. The skilled person knows how to select a polynucleotide encoding a polypeptide having a specific amino acid sequence and also knows how to optimize the codons used in the polynucleotide according to the codon usage of the organism used for expressing said polynucleotide. Thus, the term “polynucleotide variant”, as used herein relates to a variant of a polynucleotide related to herein comprising a nucleic acid sequence characterized in that the sequence can be derived from the aforementioned specific nucleic acid sequence by at least one nucleotide substitution, addition and/or deletion, wherein the polynucleotide variant shall have the activity as specified for the specific polynucleotide, i.e. shall encode a xylanase polypeptide. Preferably, said polynucleotide variant is an ortholog, a paralog or another homolog of the specific polynucleotide. Further, variants include polynucleotides comprising nucleic acid sequences which are at least 70%, at least 75%, at least 80%, at least 85%, at least 90%, at least 95%, at least 98% or at least 99% identical to the specifically indicated nucleic acid sequences. Moreover, also encompassed are polynucleotides which comprise nucleic acid sequences encoding amino acid sequences which are at least 70%, at least 75%, at least 80%, at least 85%, at least 90%, at least 95%, at least 98% or at least 99% identical to the amino acid sequences specifically indicated. The percent identity values are, preferably, calculated over the entire amino acid or nucleic acid sequence region. A series of programs based on a variety of algorithms is available to the skilled worker for comparing different sequences. In this context, the algorithms of Needleman and Wunsch or Smith and Waterman give particularly reliable results. To carry out the sequence alignments, the program PileUp (J. Mol. Evolution., 25, 351-360, 1987, Higgins et al., CABIOS, 5 1989: 151-153) or the programs Gap and BestFit (Needleman and Wunsch (J. Mol. Biol. 48; 443-453 (1970)) and Smith and Waterman (Adv. Appl. Math. 2; 482-489 (1981))], which are part of the GCG software packet (Genetics Computer Group, 575 Science Drive, Madison, Wisconsin, USA 53711 (1991)), are to be used. The sequence identity values recited above in percent (%) are to be determined, preferably, using the program GAP over the entire sequence region with the following settings: Gap Weight: 50, Length Weight: 3, Average Match: 10.000 and Average Mismatch: 0.000, which, unless otherwise specified, shall always be used as standard settings for sequence alignments.

A polynucleotide comprising a fragment of any of the specifically indicated nucleic acid sequences is also encompassed as a variant polynucleotide of the present invention. The fragment shall still encode a xylanase polypeptide as specified herein above which still has the activity as specified. Accordingly, the xylanase polypeptide encoded may comprise or consist of the domains of the xylanase polypeptide of the present invention conferring said biological activity. A fragment as meant herein, preferably, comprises at least 50, more preferably at least 100, still more preferably at least 250, most preferably at least 500, consecutive nucleotides of any one of the specific nucleic acid sequences or encodes an amino acid sequence comprising at least 50, preferably at least 100, more preferably at least 150, still more preferably at least 200, most preferably at least 200 consecutive amino acids of any one of the specific amino acid sequences. Preferably, the polynucleotide comprising a nucleic acid sequence encoding a xylanase polypeptide comprises, preferably consists of the nucleic acid sequence of any one of SEQ ID NOs:16 to 20.

The polynucleotides of the present invention either consist, essentially consist of, or comprise the aforementioned nucleic acid sequences. Thus, they may contain further nucleic acid sequences as well. Specifically, the polynucleotides of the present invention may encode fusion polypeptides wherein one partner of the fusion polypeptide is a xylanase polypeptide being encoded by a nucleic acid sequence recited above.

Preferably, the polynucleotide comprising a nucleic acid sequence encoding a xylanase polypeptide is comprised in an expression construct allowing for expression of said polynucleotide in a host cell. The term “expression construct”, as used herein, refers to a heterologous polynucleotide comprising the aforementioned polynucleotide encoding the xylanase polypeptide as well as nucleic acid sequences required for expression of the polynucleotide encoding the xylanase polypeptide.

Typically, such additional nucleic acid sequences, which preferably are heterologous to the polynucleotide encoding the xylanase polypeptide, may be promoter sequences, regulatory sequences and/or transcription termination sequences, such as terminators. Preferably, the expression construct is a eukaryotic expression construct, i.e. an expression construct comprising all elements required for expression, preferably inducible expression, in a eukaryotic host cell. More preferably, the expression construct is a prokaryotic expression construct, i.e. an expression construct comprising all elements required for expression, preferably inducible expression, in a prokaryotic host cell. Enhanced expression of the polynucleotide encoding the xylanase polypeptide may be achieved by the selection of heterologous regulatory regions, e.g. promoter, secretion leader and terminator regions, which serve to increase expression and, if desired, secretion levels of the protein of interest from the chosen expression host and/or to provide for the inducible control of the expression of the xylanase polypeptide. Aside from the promoter native to the gene encoding the xylanase polypeptide, other promoters may be used to direct expression of the polypeptide. The promoter may be selected for its efficiency in directing the expression of the xylanase polypeptide in the desired expression host. Preferably, a constitutive or inducible promoter is selected to direct the expression of the desired polypeptide. Examples of suitable promoters for directing expression of polynucleotides in a bacterial host cell are the promoters obtained from the Bacillus amyloliquefaciens alpha-amylase gene (amyQ), Bacillus licheniformis alpha-amylase gene (amyL), Bacillus licheniformis protease gene (aprE), Bacillus licheniformis penicillinase gene (penP), Bacillus stearothermophilus maltogenic amylase gene (amyM), Bacillus subtilis cytidine deaminase gene (ccd), Bacillus subtilis alpha-amylase gene (amyE), Bacillus subtilis levansucrase gene (sacB), Bacillus subtilis xylA and xylB genes, Bacillus thuringiensis cryIIIA gene (Agaisse and Lereclus, 1994, Molecular Microbiology 13: 97), E. coli lac operon, E. coli trc promoter (Egon et al., 1988, Gene 69: 301), Streptomyces coelicolor agarase gene (dagA), and prokaryotic beta-lactamase gene (Villa-Kamaroff et al., 1978, Proc. Natl. Acad. Sci. USA 75:3727), as well as the tac promoter (DeBoer et al., 1983, Proc. Natl. Acad. Sci. USA 80:21).

Further promoters are known in the art and are described in textbooks. Examples of tandem promoters are disclosed in WO 99/43835. Hybrid promoters may also be used to improve inducible regulation of the expression construct.

The control sequence may also be a transcription terminator, which is recognized by a host cell to terminate transcription. The terminator is operably linked to the 3′-terminus of the polynucleotide encoding the polypeptide. Any terminator that is functional in the host cell may be used, appropriate terminators for a given host cell are known in the art. Preferred terminators for bacterial host cells are obtained from the genes for Bacillus clausfi alkaline protease (aprH), Bacillus licheniformis alpha-amylase (amyL), Thermoactinomyces vulgaris (amyTV), and Escherichia coli ribosomal RNA (rrnB).

The control sequence may also be an mRNA stabilizer region downstream of a promoter and upstream of the coding sequence of a gene which increases expression of the gene. Examples of suitable mRNA stabilizer regions are obtained from a Bacillus thuringiensis cryIIIA gene (WO 94/25612) and a Bacillus subtilis SP82 gene (Hue et al., 1995, Journal of Bacteriology 177: 3465).

Unless specifically indicated otherwise herein, the compounds specified, in particular the polynucleotides and polypeptides, may be comprised in larger structures, e.g. may be covalently or non-covalently linked to further sequences, such as carrier or stabilizer molecules. In particular, polypeptides as specified may be comprised in fusion polypeptides comprising further amino acid sequences, which may serve e.g. as a signal peptide, as a tag for purification and/or detection, as a linker, or to extend the in vivo half-life of a compound. The term “signal peptide”, which may also be referred to as “secretion leader sequence”, “leader sequence”, or “signal sequence”, is known to the skilled person to relate a short peptide sequence present in secreted polypeptides and mediating their secretion from the cell. A signal peptide preferably is comprised in a polypeptide at its N-terminus. Preferably, the signal peptide is selected by the skilled person to concur with the host cell in which secretion shall take place; thus, a eukaryotic, preferably mammalian, signal sequence will be used for secretion from e.g. a mammalian host cell, whereas a bacterial signal peptide may be preferred for secretion from a bacterial host cell, and the like. Preferably, the signal peptide is a prokaryotic signal peptide. Prokaryotic signal peptides are known in the art and can be found in publicly available databases. Preferably, the prokaryotic signal peptide has a length of from 10 to 35 amino acids and preferably has a structure comprising an N-terminal region with a net positive charge, followed by a central hydrophobic region, followed by a C-terminal polar region, the C-terminal polar region preferably including a signal peptidase recognition motif.

Often, it is desirable for the polypeptide to be secreted from the expression host into the culture medium from where the polypeptide of the invention may be more easily recovered. Preferably, the native signal sequence of the xylanase polypeptide is used to effect the secretion of the polypeptide of the invention. However, an increase in the production of a polypeptide may result in the production of the polypeptide in levels beyond that which the host is capable of processing and secreting, creating a bottleneck such that the protein product accumulates within the cell. Accordingly, a heterologous leader sequence may be used to provide for the most efficient secretion of the polypeptide from the chosen expression host. The secretion leader may be selected on the basis of the desired expression host. A heterologous secretion leader may be chosen which is homologous or heterologous to the other regulatory regions of the expression construct. For example, the leader of the highly secreted amyloglucosidase (AG) protein may be used in combination with the amyloglucosidase (AG) promoter itself, as well as in combination with other promoters. Hybrid signal sequences may also be used with the context of the present invention. Effective signal peptide coding sequences for bacterial host cells are the signal peptide coding sequences obtained from the genes for Bacillus NCIB 11837 maltogenic amylase, Bacillus licheniformis subtilisin, Bacillus licheniformis beta-lactamase, Bacillus stearothermophilus alpha-amylase, Bacillus stearothermophilus neutral proteases (nprT, nprS, nprM), and Bacillus subtilis prsA. Further signal peptides are described by Simonen and Palva, 1993, Microbiological Reviews 57:109.

Preferably, the signal peptide is a signal peptide from a gram-positive bacterium, in particular in case the polypeptide is expressed in a gram positive bacterium, e.g. a Bacillus spec. Preferably, the sequence of the signal peptide is a sequence selected from the list consisting of SEQ ID NO:7 or a sequence at least 70% identical to SEQ ID NO:7, more preferably a sequence essentially identical to SEQ ID NO:7, most preferably the sequence of SEQ ID NO:7

Preferably, the polynucleotide comprising a nucleic acid sequence encoding a xylanase polypeptide, more preferably the expression construct comprising the polynucleotide comprising a nucleic acid sequence encoding a xylanase polypeptide, is comprised in a vector.

The term “vector”, as used herein, relates to any polynucleotide adapted for stably maintaining the polynucleotide and/or the expression construct as specified herein above in a host cell. The term vector preferably encompasses phage, plasmid, and viral vectors as well artificial chromosomes, such as bacterial or yeast artificial chromosomes. Preferably, the vector is a plasmid, preferably a linear or closed circular plasmid, more preferably a closed circular plasmid. The vector preferably is a vector (e.g., a plasmid, phage or virus) that can be conveniently subjected to recombinant DNA procedures and can preferably bring about expression of the polynucleotide, i.e., preferably is an expression vector. The choice of the vector will typically depend on the compatibility of the vector with the host cell into which the vector is to be introduced. The vector may be a linear or closed circular plasmid. The vector may be an autonomously replicating vector, i.e., a vector that exists as an extrachromosomal entity, the replication of which is independent of chromosomal replication, e.g., a plasmid, an extrachromosomal element, a minichromosome, or an artificial chromosome. The vector may contain any means for assuring self-replication. Alternatively, the vector may be one that, when introduced into the host cell, is integrated into the genome and replicated together with the chromosome(s) into which it has been integrated. Furthermore, a single vector or plasmid or two or more vectors or plasmids that together contain the total DNA to be introduced into the genome of the host cell, or a transposon, may be used. Alternatively, CRISPR-Cas technology can be used in introduction of a polynucleotide, an expression construct, and/or a vector as specified herein into the genome of the host cell. The vector preferably contains one or more selectable markers that permit easy selection of transformed, transfected, transduced, or the like cells. A selectable marker is a gene the product of which provides for biocide or viral resistance, resistance to heavy metals, prototrophy to auxotrophs, and the like. Examples of bacterial selectable markers are Bacillus subtilis, Bacillus amyloliquefaciens or Bacillus licheniformis gpsA, pyrF or dal genes, or markers that confer antibiotic resistance such as ampicillin, chloramphenicol, kanamycin, neomycin, spectinomycin, or tetracycline resistance. The vector preferably contains an element(s) that permits integration of the vector into the host cell's genome or autonomous replication of the vector in the cell independent of the genome.

For integration into the host cell genome, the vector may rely on the polynucleotide's sequence encoding the polypeptide or any other element of the vector for integration into the genome by homologous or non-homologous recombination. Alternatively, the vector may contain additional polynucleotides for directing integration by homologous recombination into the genome of the host cell at a precise location(s) in the chromosome(s). To increase the likelihood of integration at a precise location, the integrational elements preferably contain a sufficient length of nucleic acid sequence, such as 100 to 10,000 base pairs, 400 to 10,000 base pairs, and 800 to 10,000 base pairs, which has a high degree of sequence identity to the corresponding target sequence to enhance the probability of homologous recombination. The integrational elements may be any sequence that is homologous with the target sequence in the genome of the host cell. Furthermore, the integrational elements may be non-encoding or encoding polynucleotides. On the other hand, the vector may be integrated into the genome of the host cell by non-homologous recombination. For autonomous replication, the vector may further comprise an origin of replication enabling the vector to replicate autonomously in the host cell in question. The origin of replication may be any plasmid replicator mediating autonomous replication that functions in a cell. The term “origin of replication” or “plasmid replicator” means a polynucleotide that enables a plasmid or vector to replicate in vivo.

Examples of bacterial origins of replication are the origins of replication of plasmids pBR322, pUC19, pACYC177, and pACYC184 permitting replication in E. coli, and pUB110, pE194, pTA1050, and pAMR1 permitting replication in Bacillus.

The vector may be incorporated into a host cell by various techniques well known in the art. For example, a plasmid vector can be introduced in a precipitate such as a calcium phosphate precipitate or rubidium chloride precipitate, or in a complex with a charged lipid or in carbon-based clusters, such as fullerenes. Alternatively, a plasmid vector may be introduced by heat shock or electroporation techniques. Should the vector be a virus, including phages, it may be packaged in vitro using an appropriate packaging cell line prior to application to host cells. As indicated in more detail elsewhere herein, the host cell can be a prokaryotic or a eukaryotic cell.

Preferably, the vector is a bacterial vector, preferably a Bacillus vector, in particular a Bacillus plasmid. Suitable vectors are in principle known in the art and are described in textbooks. Preferably, the vector is an expression vector and/or a gene transfer or targeting vector. Methods which are well known to those skilled in the art can be used to construct recombinant polynucleotides and vectors and to transfer such recombinant constructs into host cells; see, for example, the techniques described in Sambrook et al (Molecular Cloning: A Laboratory Manual, 2nd edition, 1989, Cold Spring Harbor Laboratory Press) and Ausubel et al., Short Protocols in Molecular Biology (1999), 4th Ed., John Wiley & Sons, Inc.

As used herein, the term “host cell” relates to any cell capable of receiving and, preferably maintaining, the polynucleotide as specified herein above. More preferably, the host cell is capable of expressing a xylanase polypeptide encoded on the expression polynucleotide and/or expression vector as specified herein above. Preferably, the host cell is a eukaryotic cell, preferably a fungal cell, e.g. a cell of Trichoderma reesei, Trichoderma viride, Trichoderma harzianum, Aspergillus niger, Aspergillus oryzae, Humicola insolens, or Humicola grisea, or a strain of baker's yeast. The host cell may, however, also be an animal cell, e.g. an insect cell or a mammalian cell. More preferably, the host cell is a bacterial cell, i.e. a prokaryotic cell, more preferably a gram-positive or gram-negative bacterial cell. Preferably, the host cell is a gram-negative host cell, preferably of a strain commonly used in industry, preferably food industry, or in the laboratory, e.g. an E. coli cell. More preferably, the host cell is a gram-positive host cell, preferably of a strain commonly used in industry, preferably food industry, or in the laboratory; thus, more preferably, the host cell is a cell of the genus Bacillus, in particular Bacillus subtilis, Bacillus pumilus, Bacillus alkalophilus, Bacillus amyloliquefaciens, Bacillus brevis, Bacillus circulans, Bacillus clausii, Bacillus coagulans, Bacillus halodurans, Bacillus lautus, Bacillus lentus, Bacillus licheniformis, Bacillus megaterium, Bacillus stearothermophilus, or Bacillus thuringiensis. Most preferably, the host cell is a cell of B. subtilis or B. pumilus.

In view of the above, the instant invention also relates to a gram-positive bacterial host cell comprising (I) a xylanase polypeptide comprising an amino acid sequence at least 70% identical to SEQ ID NO:1 and an amino acid exchange at the position corresponding to amino acid S22 in SEQ ID NO:1 and/or (II) a polynucleotide encoding the xylanase polypeptide of (I).

The xylanase polypeptide comprising an amino acid sequence at least 70% identical to SEQ ID NO:1 and an amino acid exchange at the position corresponding to amino acid S22 in SEQ ID NO:1, preferably, comprises, more preferably consists of, the amino acid sequence of SEQ ID NO:2 or a sequence at least 90%, preferably at least 95%, more preferably at least 98%, most preferably at least 99% identical thereto. More preferably, the xylanase polypeptide comprises, more preferably consists of, the amino acid sequence of SEQ ID NO:2. Also preferably, the xylanase polypeptide comprises, more preferably consists of, the amino acid sequence of SEQ ID NO:9 or a sequence at least 90%, preferably at least 95%, more preferably at least 98%, most preferably at least 99% identical thereto. More preferably, the xylanase polypeptide comprises, more preferably consists of, the amino acid sequence of SEQ ID NO:9. More preferably, the xylanase comprised in the gram-positive bacterial host cell is a xylanase polypeptide as specified herein above, in particular comprising, preferably consisting of, the amino acid sequence of any one of SEQ ID NOs:3 to 6, more preferably any one of SEQ ID NOs:10 to 13. Preferably, said gram-positive bacterial host cell is gram-positive bacterial host cell as specified herein above, in particular a Bacillus cell.

The present invention further relates to a method of producing a xylanase polypeptide comprising expressing a polynucleotide of the present invention in a host cell.

The method of producing a xylanase polypeptide of the present invention is an in vitro method. Moreover, it may comprise steps in addition to those explicitly mentioned above. For example, further steps may relate, e.g., to providing a host cell comprising a polynucleotide as specified, preferably an expression construct, pre-growing the host cells before producing the xylanase polypeptide, and/or one or more steps of purifying the xylanase polypeptide. Moreover, one or more of said steps may be assisted or performed by automated equipment.

The term “expressing a polynucleotide” is understood by the skilled person. Preferably, expressing a polynucleotide comprises incubating a host cell comprising the polynucleotide, more preferably an expression construct comprising the polynucleotide, under conditions permitting the host cell to produce a polypeptide encoded by said polynucleotide. The host cells are preferably cultivated in a nutrient medium suitable for production of the polypeptide using methods known in the art. For example, the cells may be cultivated by shake flask cultivation, or small-scale or large-scale fermentation (including continuous, batch, fed-batch, or solid state fermentations) in laboratory or industrial fermenters in a suitable medium and under conditions allowing the polypeptide to be expressed and/or isolated. The cultivation preferably takes place in a suitable nutrient medium comprising carbon and nitrogen sources and inorganic salts, using procedures known in the art. Suitable media are available from commercial suppliers or may be prepared according to published compositions (e.g., in catalogues of the American Type Culture Collection).

The polypeptide may be detected using methods known in the art that are specific for the polypeptides. These detection methods include, but are not limited to, use of specific antibodies, formation of an enzyme product, or disappearance of an enzyme substrate. For example, an enzyme assay may be used to determine the activity of the polypeptide, e.g. a xylanase assay as specified herein above and in the Examples. If the polypeptide is secreted into the nutrient medium, the polypeptide can be recovered directly from the medium. If the polypeptide is not secreted, it can be recovered from cell lysates. The polypeptide may be recovered using methods known in the art. For example, the polypeptide may be recovered from the nutrient medium by conventional procedures including, but not limited to, collection, centrifugation, filtration, extraction, spray-drying, evaporation, or precipitation. In one aspect, a fermentation broth comprising the polypeptide is recovered. The polypeptide may be purified by a variety of procedures known in the art including, but not limited to, chromatography (e.g., ion exchange, affinity, hydrophobic, chromatofocusing, and size exclusion), electrophoretic procedures (e.g., preparative isoelectric focusing), differential solubility (e.g., ammonium sulfate precipitation), SDS-PAGE, or extraction (see, e.g., Protein Purification, Janson and Ryden, editors, VCH Publishers, New York, 1989) to obtain essentially pure polypeptides.

Preferably, the polypeptide is not recovered, but rather a host cell of the present invention expressing the polypeptide is used as a source of the polypeptide. Thus, the invention also relates to a method for producing a xylanase polypeptide, said method comprising: a) cultivating a Bacillus host cell in a medium and under conditions conducive for the production of said polypeptide; and optionally b) recovering said xylanase polypeptide. Said conditions preferably comprise incubation of the host cell in a culture medium, preferably a liquid culture medium, at a temperature suitable for the host cell to express a polynucleotide. Preferred temperatures and culture media depend on the host cell selected and are known in the art. Preferably, in case the host cell is a Bacillus spec., the culture medium is 1-10% carbon source, 5-8% complex nitrogen source, salts, pH 6.5-8 and/or the temperature is of from 4° C. to 50° C., preferably of from 10° C. to 45° C., more preferably of from 20° C. to 40° C. Preferably, in particular in case Bacillus spec. is a Bacillus pumilus, the culture medium is 2% carbon source, 6% complex nitrogen source, salts, pH 7.5, and the temperature is 37° C.; or in particular in case the Bacillus spec. is a Bacillus subtilis, the culture medium is 4% carbon source, 7% complex nitrogen source, salts, pH 7.0, and the temperature is 37° C.

Preferably, the method of producing a xylanase polypeptide further comprises pre-culturing the host cells. Thus, in particular in case an inducible expression system is used, the host cells preferably are pre-grown to a pre-defined cell density before inducing expression of the polynucleotide. The conditions during pre-culturing may be the same or essentially the same during pre-culture and expression culture; they may, however, also be different, e.g. pre-culture may be performed at a temperature optimal for host cell growth, while expression may be performed at a temperature optimal for polynucleotide expression and/or polypeptide production.

Preferably, the method of producing a xylanase polypeptide further comprises at least one step of purifying the xylanase polypeptide. The xylanase polypeptide may, depending on the host cell and the expression construct used, be purified from the supernatant of the culture medium after removal on the host cell, or from the host cells, or both. As will be understood, in case of purification from the host cells, the host cells are preferably lysed. In particular in case the host cell is a Bacillus spec. and in case the xylanase polypeptide is expressed including a signal peptide, the xylanase polypeptide preferably is obtained, more preferably purified, from the supernatant of the culture medium. Preferably, in such case the culture supernatant is filtered to remove cells and/or cell fragments. Preferably, the purification at least comprises at least partially separating the xylanase polypeptide from low-molecular weight compounds having a molecular mass of less than 1 kDa comprised in the growth medium, such as salts, carbon sources, buffer compounds, and the like. Preferably, the xylanase polypeptide is purified to remove other polysaccharide-hydrolyzing activities. More preferably, the xylanase polypeptide is purified to at least 80%, more preferably at least 90%, even more preferably at least 95% purity, as estimated from an SDS-PAGE stained with CoomassieBlue.

Preferably, the xylanase polypeptide is produced with a signal peptide according to the method of producing a xylanase polypeptide. Thus, the polynucleotide expressed preferably encodes a xylanase polypeptide including a signal sequence, both as specified herein above. Thus, the polynucleotide preferably encodes a polynucleotide comprising the amino acid sequence of any one of SEQ ID NOs:9 to 13. More preferably, the polynucleotide comprises the nucleic acid sequence of any one of SEQ ID NOs: 16 to 20. As the skilled person will understand, the signal peptide will be removed by the host cell during transport of the xylanase polypeptide to the exterior of the cell, thus, the xylanase polypeptide produced in a host cell with a signal peptide will preferably not comprise a signal peptide once transferred by the host cell into the culture medium.

The present invention also relates to a method of producing a bakery product comprising (a) admixing a xylanase polypeptide according to the present invention to flour and water, and (b) incubating the admixture of step (a) for an incubation period.

The method of producing a bakery product may comprise steps in addition to those mentioned above; also, one or more steps may be assisted of performed by automated equipment. Exemplary further steps may relate to kneading and/or incubating the admixture for one or more further incubation period(s). E.g. the method may comprise production of a pre-dough according to the method as specified herein above, followed by kneading and optionally addition of further components, and optionally followed by a second incubation. The method as specified may, however, also be preceded by the production of a pre-dough, to which components as specified for step (a) are admixed. Preferably, the method comprises further step (c) baking the dough obtained in step (b). Also preferably, further components may be admixed to the bakery product, in particular additives as specified herein below.

The term “additive”, as used herein, relates to any compound used for improving properties of a dough and/or a baked good, such as an oxidant, an antioxidant, a hydrocolloid, a preservative, and/or a bread enzyme; and or any compound stabilizing and/or improving handling of the xylanase polypeptide, such as and antioxidant or an inert dilution compound, such as starch. Preferred enzymes for use as additive are one or more enzymes selected from the group consisting of amylase, maltogenic amylase, beta amylase, aminopeptidase, carboxypeptidase, catalase, cellulase, chitinase, cutinase, cyclodextrin glycosyltransferase, deoxyribonuclease, esterase, glucan 1,4-alpha-maltotetrahydrolase, glucanase, galactanase, alpha-galactosidase, beta-galactosidase, glucoamylase, glucose oxidase, alpha-glucosidase, beta-glucosidase, haloperoxidase, hemicellulytic enzyme, invertase, laccase, lipase, mannanase, mannosidase, oxidase, pectinolytic enzymes, peptidoglutaminase, peroxidase, phospholipase, phytase, polyphenoloxidase, protease, ribonuclease, transglutaminase, and xylanase.

The term “bakery product”, as used herein, includes any and all products produced by a method including obtaining an admixture of flour and water. Thus, the bakery product preferably is a dough or a baked good as specified herein below.

The terms “flour” and “water” are known to the skilled person. Preferably, the flour is a powder generated by grinding grains, roots, beans, nuts, or other fruits of edible plants or parts thereof. Preferably, the flour is cereal flour, more preferably wheat, rye, barley, oat, corn, rice, spelt, sorghum, millet, emmer, einkorn, kamut, or buckwheat flour, more preferably is wheat flour. The flour may be any type and may have any residual ash mass deemed appropriate by the skilled person; preferably, the flour is pastry flour, all-purpose flour, or bread flour. The water, preferably, is food-grade water.

The term “dough”, is used herein in a broad sense relating to any and all mixtures comprising the indicated compounds. Preferably, the dough comprises at least 10% (w/w) flour, more preferably at least 20% (w/w) flour, still more preferably at least 30% (w/w) flour, even more preferably at least 40% (w/w) flour, most preferably at least 50% (w/w) flour. Thus, the dough may be a liquid dough such as a batter or a semisolid or solid dough, preferably is a semisolid or solid dough. The dough comprises at least one type of flour, more preferably comprises one type of flour; thus, the dough may also comprise different types of flour, both with regards to the source organism of the flour and to the flour type; thus, as non-limiting examples, the dough may e.g. be a mixed wheat flour/rye flour dough, and/or may be a mixed whole grain/bread flour dough. Optionally, the dough may comprise a bread improver or other bread ingredient, preferably selected from bread enzymes as specified herein above, salts (e.g. sodium chloride, an acetate salt, a fumarate salt, a citrate salt, and/or a carbonate salt), and/or antioxidants (e.g. ascorbic acid, or ascorbate salts). Preferably, the dough and/or baked goods produced therefrom are for human or animal food consumption, are human food or animal feed. Preferably, the dough and/or baked goods produced therefrom are food, i.e. for human consumption. Preferably, the dough produced according to the aforesaid method has improved properties, in particular decreased stickiness compared to a dough produced with a xylanase polypeptide consisting of the amino acid sequence of SEQ ID NO:1 or 14 under otherwise essentially identical conditions. Also preferably, said dough has an increased softness compared to a dough produced with a xylanase polypeptide consisting of the amino acid sequence of SEQ ID NO:1 or 14 under otherwise essentially identical conditions.

The term “baked good” includes any and all goods made from a dough or batter and cooked by heating, in particular by baking, frying, or deep frying, more preferably by baking. Preferably, the baked good is a bread, including flatbreads, bagels, breadrolls, and the like; a cracker; a pastry product; a tart or pie; or a viennoiserie. Preferably, the baked good is made from an unleavened dough, i.e. lacking added yeast, e.g. in case of an unleavened flatbread such as tortilla, chapati, dosa, and the like. More preferably, the baked good is made from a leavened dough, such as loaf bread such as toast, baguette, and the like; leavened flatbread; or viennoiserie. Even more preferably, the baked good is a loaf bread, more preferably a bread comprising at least 10% wheat flour of the total amount of flour, even more preferably is a wheat bread. Preferably, the baked good has a crust, i.e. preferably is a bun, a loaf of bread, a whole-grain loaf, a bread roll, a pretzel, or a pizza. Preferably, the baked good has an increased volume compared to a baked good produced with a xylanase polypeptide consisting of the amino acid sequence of SEQ ID NO:1 or 14 under otherwise essentially identical conditions.

The present invention also relates to a composition comprising a xylanase polypeptide according to the present invention and at least one additive and/or a at least one flour.

The term “composition”, as used herein, relates to any composition of matter comprising the components as indicated. Preferably, the composition is a dough improver, preferably a wheat dough improver. Thus, the composition has the activity of improving properties of a dough, in particular processing properties of a dough. More preferably, the composition has the activity of improving properties of a dough in the presence of a xylanase inhibitor at a concentration typically present in a dough. Preferably, the composition has the activity of improving stickiness after dough mixing, stickiness after resting (i.e. after an incubation period), and of increasing the volume of a baked good produced therefrom. The composition may be a liquid preparation or a solid preparation. Thus, the xylanase polypeptide may be comprised in the composition as a solubilized polypeptide, or may be comprised in the composition in a dry form, e.g. as a lyophilized or spray-dried preparation. The composition may be a ready-to-use improvement mixture for a bakery good comprising the xylanase polypeptide and one or more bread improver known in the art. The composition may, however, also be a stabilized preparation of the xylanase polypeptide.

Preferably, the composition further comprises a flour, preferably as specified herein above, and/or comprises water, also preferably as specified herein above. Thus, the composition may be a bakery product, in particular a dough or a baked good.

In accordance with the above, the present invention also relates to a bakery product produced or producible according to the method of producing a bakery product of the present invention and/or comprising a composition comprising a xylanase polypeptide of the present invention.

Further, the present invention relates to a use of a xylanase polypeptide according to the present invention for hydrolyzing xylan, preferably in a dough and to a use of a polynucleotide according to the present invention for producing a xylanase polypeptide. In accordance, the present invention also relates to a use of a xylanase polypeptide according to the present invention e.g. in the preparation of foodstuff, preferably a bakery product.

As the skilled person understands, there are many applications of xylanases, i.e. processes requiring or being improved by hydrolyzing xylan.

Thus, a xylanase polypeptide may be used to process plant materials such as cereals that are used in foodstuffs including animal feed. As used herein, the term “cereal” means any kind of grain used for food and/or any grass producing this grain such as but not limited to any one of wheat, milled wheat, barley, maize, sorghum, rye, oats, triticale and rice or combinations thereof. In one preferred embodiment, the cereal is a wheat cereal. The xylan in the food and/or feed supplement preferably is modified by contacting the xylan with the variant xylanase polypeptide. As used herein, the term “contacting” includes but is not limited to spraying, coating, impregnating, layering, or admixing the food and/or feed supplement with the variant xylanase polypeptide. The food and/or feed supplement may be prepared by mixing the xylanase polypeptide directly with a food and/or feed supplement. By way of example, the xylanase polypeptide may be contacted (for example, by spraying) onto a cereal-based food and/or feed supplement such as milled wheat, maize or soya flour. It is also possible to incorporate the xylanase polypeptide into a second (and different) food and/or feed or drinking water which is then added to the food and/or feed supplement. Accordingly, it is not essential that the variant xylanase enzyme provided by the present invention is incorporated into the cereal-based food and/or feed supplement itself, although such incorporation forms a particularly preferred aspect. The food and/or feed supplement may be combined with other food and/or feed components to produce a cereal-based food and/or feed. Such other food and/or feed components may include one or more other (preferably thermostable) enzyme supplements, vitamin food and/or feed supplements, mineral food and/or feed supplements and amino acid food and/or feed supplements. The resulting (combined) food and/or feed supplement comprising possibly several different types of compounds can then be mixed in an appropriate amount with the other food and/or feed components such as cereal and protein supplements to form a human food and/or an animal feed.

The food and/or feed supplement can be prepared by mixing different enzymes having the appropriate activities to produce an enzyme mix. By way of example, a cereal-based food and/or feed supplement formed from e.g. milled wheat or maize may be contacted (e.g. by spraying) either simultaneously or sequentially with the xylanase polypeptide and other enzymes having appropriate activities. These enzymes may include but are not limited to any one or more of an amylase, a glucoamylase, a mannanase, a galactosidase, a phytase, a lipase, a glucanase, an arabinofuranosidase, a pectinase, a protease, a glucose oxidase, a hexose oxidase and a xylanase. Enzymes having the desired activities may for instance be mixed with the xylanase polypeptide either before contacting these enzymes with a cereal-based food and/or feed supplement or alternatively such enzymes may be contacted simultaneously or sequentially on such a cereal based supplement. The food and/or feed supplement is then in turn mixed with a cereal-based food and/or feed to prepare the final food and/or feed. It is also possible to formulate the food and/or feed supplement as a solution of the individual enzyme activities and then mix this solution with a food and/or feed material prior to processing the food and/or feed supplement into pellets or as a mash.

Also described herein is the use of a xylanase polypeptide in a process for preparing a foodstuff. Typical bakery (baked) products in accordance with the present invention include bread—such as loaves, rolls, buns, pizza bases etc.—pretzels, tortillas, cakes, cookies, biscuits, crackers etc. The preparation of foodstuffs such as bakery products is well known in the art. A xylanase polypeptide of the invention may also be used in starch production from plant materials derived from cereals and tubers, such as potatoes. A xylanase polypeptide may also be used in processing wood pulp, for example in the preparation of paper.

Furthermore, the present invention relates to a kit comprising a xylanase polypeptide according to the present invention, a polynucleotide according to the present invention, and/or a host cell according to the present invention, comprised in a housing.

The term “kit”, as used herein, refers to a collection of the aforementioned compounds, means or reagents. The components of the kit may be comprised by separate vials (i.e. as a kit of separate parts) or provided in a single vial, e.g. as a composition as specified herein above. The housing of the kit preferably allows translocation of the compounds of the kit, in particular common translocation; thus, the housing may in particular be a transportable container comprising all specified components. Moreover, it is to be understood that the kit of the present invention may be used for practicing at least one of the methods referred to herein above. It is envisaged that all components may be provided in a ready-to-use manner for practicing a method referred to above. Further, the kit preferably contains instructions for carrying out said methods, e.g. dosage instructions. The instructions can be provided by a user's manual in paper- or electronic form.

The present invention further relates to a method of producing a composition according to the present invention, comprising admixing a xylanase polypeptide according to the present invention to at least one additive and/or at least one flour.

In view of the above, the following embodiments are particularly envisaged:

Embodiment 1: A xylanase polypeptide comprising
(i) an amino acid sequence at least 70% identical to SEQ ID NO:1,
(ii) an amino acid exchange at the position corresponding to amino acid D11 in SEQ ID NO:1; and
(iii) an amino acid exchange at the position corresponding to amino acid S22 in SEQ ID NO:1.

Embodiment 2: The xylanase polypeptide of embodiment 1, wherein said amino acid exchange at the position corresponding to amino acid D11 in SEQ ID NO:1 is a D11Y, a D11F, a D11K, a D11N, a D11K, a D11S, or a D11W exchange.

Embodiment 3: The xylanase polypeptide of embodiment 1 or 2, wherein said amino acid exchange at the position corresponding to amino acid D11 in SEQ ID NO:1 is a D11Y, a D11F, or a D11K exchange.

Embodiment 4: The xylanase polypeptide of any one of embodiments 1 to 3, wherein said amino acid exchange at the position corresponding to amino acid D11 in SEQ ID NO:1 is a D11Y or a D11F exchange.

Embodiment 5: The xylanase polypeptide of any one of embodiments 1 to 4, wherein said amino acid exchange at the position corresponding to amino acid S22 in SEQ ID NO:1 is an S22E, an S22D, an S22N, an S22Q, an S22A, an S22V, an S22L, or an S22I exchange.

Embodiment 6: The xylanase polypeptide of any one of embodiments 1 to 5, wherein said amino acid exchange at the position corresponding to amino acid S22 in SEQ ID NO:1 is an S22E or an S22D exchange.

Embodiment 7: The xylanase polypeptide of any one of embodiments 1 to 6, wherein said amino acid exchange at the position corresponding to amino acid S22 in SEQ ID NO:1 is an S22E exchange.

Embodiment 8: The xylanase polypeptide of any one of embodiments 1 to 7, wherein said xylanase polypeptide further comprises an amino acid exchange at the position corresponding to amino acid R122 in SEQ ID NO:1.

Embodiment 9: The xylanase polypeptide of any one of embodiments 1 to 8, wherein said amino acid exchange at the position corresponding to amino acid R122 in SEQ ID NO:1 is an R122D, an R122N, an R122E, an R122Y, an R122F, an R122K, or an R122A exchange.

Embodiment 10: The xylanase polypeptide of any one of embodiments 1 to 9, wherein said amino acid exchange at the position corresponding to amino acid R122 in SEQ ID NO:1 is an R122D or R122N exchange.

Embodiment 11: The xylanase polypeptide of any one of embodiments 1 to 10, wherein said xylanase polypeptide further comprises an amino acid exchange at the position corresponding to amino acid G34 in SEQ ID NO:1.

Embodiment 12: The xylanase polypeptide of any one of embodiments 1 to 11, wherein said amino acid exchange at the position corresponding to amino acid G34 in SEQ ID NO:1 is a G34D exchange.

Embodiment 13: The xylanase polypeptide of any one of embodiments 1 to 12, wherein said amino acid sequence of the xylanase polypeptide is at least 80%, preferably at least 90%, more preferably at least 95%, most preferably at least 98%, identical to SEQ ID NO:1.

Embodiment 14: The xylanase polypeptide of any one of embodiments 1 to 13, wherein said xylanase polypeptide comprises, preferably consists of, the amino acid sequence of SEQ ID NO:3, 4, 10, or 11 or a sequence at least 90%, preferably at least 95%, more preferably at least 98%, most preferably at least 99% identical thereto.

Embodiment 15: The xylanase polypeptide of any one of embodiments 1 to 14, wherein said xylanase polypeptide comprises, preferably consists of, the amino acid sequence of SEQ ID NO:5, 6, 12, or 13 or a sequence at least 90%, preferably at least 95%, more preferably at least 98%, most preferably at least 99% identical thereto.

Embodiment 16: The xylanase polypeptide of any one of embodiments 1 to 15, wherein said xylanase polypeptide comprises, preferably consists of, the amino acid sequence of SEQ ID NO:3 or 5, preferably 5.

Embodiment 17: The xylanase polypeptide of any one of embodiments 1 to 16, wherein said xylanase polypeptide has a xylanase activity, preferably endo-β-1,4-xylanase activity (EC 3.2.1.8).

Embodiment 18: The xylanase polypeptide of any one of embodiments 1 to 17, wherein said xylanase polypeptide has a xylanase activity which is essentially not inhibited by a wheat endogenous xylanase inhibitor.

Embodiment 19: The xylanase polypeptide of any one of embodiments 1 to 18, wherein said xylanase polypeptide has a xylanase activity which is essentially not inhibited by a wheat endogenous xylanase inhibitor at a concentration of 10 xylanase inhibitor units/mL.

Embodiment 20: The xylanase polypeptide of any one of embodiments 1 to 19, wherein said xylanase polypeptide is producible at an at least 1.3-fold increased volume yield compared to a xylanase polypeptide consisting of the amino acid sequence of SEQ ID NO:1 or 14 produced and purified under essentially the same conditions and/or having essentially the same purity.

Embodiment 21: The xylanase polypeptide of any one of embodiments 1 to 20, wherein said xylanase polypeptide is producible in a prokaryotic expression system at an at least twofold volume yield compared to a xylanase polypeptide consisting of the amino acid sequence of SEQ ID NO:4 produced under essentially the same conditions.

Embodiment 22: A polynucleotide comprising a nucleic acid sequence encoding a xylanase polypeptide according to any one of embodiments 1 to 21.

Embodiment 23: The polynucleotide of embodiment 22, wherein said polynucleotide is comprised in an expression construct.

Embodiment 24: The polynucleotide of embodiment 22 or 23, wherein said polynucleotide is comprised in a vector.

Embodiment 25: A host cell comprising the xylanase polypeptide according to any one of embodiments 1 to 21 and/or the polynucleotide according to any one of embodiments 22 to 24.

Embodiment 26: The host cell of embodiment 25, wherein said host cell is a bacterial cell, preferably a gram-positive bacterial cell.

Embodiment 27: The host cell of embodiment 25 or 26, wherein said host cell is a bacterial cell of the genus Bacillus, preferably a B. pumilus or a B. subtilis cell.

Embodiment 28: A gram-positive bacterial host cell comprising (I) a xylanase polypeptide comprising an amino acid sequence at least 70% identical to SEQ ID NO:1 and an amino acid exchange at the position corresponding to amino acid S22 in SEQ ID NO:1 and/or (II) a polynucleotide encoding the xylanase polypeptide of (I).

Embodiment 29: A method of producing a xylanase polypeptide comprising expressing a polynucleotide according to any one of embodiments 22 to 24 in a host cell.

Embodiment 30: The method of embodiment 29, wherein said method further comprises at least one step of purifying said xylanase polypeptide.

Embodiment 31: A method of producing a bakery product comprising
(a) admixing a xylanase polypeptide according to any one of embodiments 1 to 21 to flour and water, and
(b) incubating the admixture of step (a) for an incubation period.

Embodiment 32: The method of embodiment 31, wherein said bakery product is a dough or a baked good.

Embodiment 33: The method of embodiment 32, wherein said dough has a decreased stickiness compared to a dough produced with a xylanase polypeptide consisting of the amino acid sequence of SEQ ID NO:1 or 14 under otherwise essentially identical conditions.

Embodiment 34: The method of embodiment 32 or 33, wherein said dough has an increased softness compared to a dough produced with a xylanase polypeptide consisting of the amino acid sequence of SEQ ID NO:1 or 14 under otherwise essentially identical conditions.

Embodiment 35: The method of any one of embodiments 31 to 34, wherein said method comprises further step (c) baking the dough obtained in step (b).

Embodiment 36: The method of any one of embodiments 32 to 35, wherein said bakery product is a baked good, preferably a bread.

Embodiment 37: The method of any one of embodiments 32 to 36, wherein said bakery product has an increased volume and/or decreased dough stickiness compared to a bakery product produced with a xylanase polypeptide consisting of the amino acid sequence of SEQ ID NO:1 or 4 under otherwise essentially identical conditions.

Embodiment 38: A composition comprising a xylanase polypeptide according to any one of embodiments 1 to 21 and at least one additive and/or at least one flour.

Embodiment 39: The composition of embodiment 38, wherein said composition is a dough improver, preferably a wheat dough improver.

Embodiment 40: The composition of embodiment 38 or 39, wherein said composition is a ready-to-use flour.

Embodiment 41: The composition of any one of embodiments 38 to 40, wherein said composition further comprises water.

Embodiment 42: The composition of any one of embodiments 38 to 41, wherein said composition is a dough or a baked good.

Embodiment 43: A bakery product produced or producible according to the method according to any one of embodiments 31 to 37 and/or comprising a composition according to any one of embodiments 38 to 42.

Embodiment 44: Use of a xylanase polypeptide according to any one of embodiments 1 to 21 for hydrolyzing xylan, preferably in a dough.

Embodiment 45: Use of a polynucleotide according to any one of embodiments 22 to 24 for producing a xylanase polypeptide.

Embodiment 46: A kit comprising a xylanase polypeptide according to any one of embodiments 1 to 21, a polynucleotide according to any one of embodiments 22 to 24, and/or a host cell according to any one of embodiments 25 to 28, comprised in a housing.

Embodiment 47: A method of producing a composition according to any one of embodiments 38 to 42, comprising admixing a xylanase polypeptide according to any one of embodiments 1 to 21 to at least one additive and/or at least one flour.

All references cited in this specification are herewith incorporated by reference with respect to their entire disclosure content and the disclosure content specifically mentioned in this specification.

### FIGURE LEGENDS

FIG. 1: Increased production in Bacillus subtilis of xylanase variants containing amino acid exchange S22E in comparison to the parent enzyme shown by xylanase activity (XylH).

FIG. 2: Increased production in Bacillus pumilus of xylanase variants containing amino acid exchange S22E in comparison to the parent enzyme shown by protein content in (g/L) and by xylanase activity (XylH).

FIG. 3: Increased production in Bacillus subtilis and Bacillus pumilus of xylanase variants containing amino acid exchange S22E in comparison to the parent enzyme shown by SDS Page.

The following Examples shall merely illustrate the invention. They shall not be construed, whatsoever, to limit the scope of the invention.

### EXAMPLE 1: MEASURING XYLANASE ACTIVITY

### Principle

The xylan fragments released by enzymatic hydrolysis of xylan are determined photometrically at 412 nm using p-hydroxybenzoic acid hydrazide (PAHBAH).

### Unit of Activity

One Xylh unit corresponds to that enzyme quantity which releases under standard conditions 1 mol xylose by hydrolysis of xylan in one minute at 30° C. The unit of activity is Xylh/g.

### Assay

0.75 ml of substrate solution was added to test tubes and incubated at 30° C. for a minimum of 5 minutes. The reaction was started by adding 0.25 ml of sample and mix. After an incubation period of exactly 20 min., the reaction was stopped by adding 4.0 ml of hydrazide reagent. Test tubes were incubated 30 min at 75° C. Test tubes were cooled in cold water for about 10 min and the sample absorbance was measured against the reagent blank.

Reagent blank contained 1.0 ml of 40 mM acetate buffer and 6.0 ml hydrazide reagent and was handled at 75° C. as the samples. Enzyme blank: First add to substrate solution 4.0 ml of hydrazide reagent and then 0.25 ml of sample. Do not incubate at 30° C., but handle as samples in 75° C.

### Substrate Solution

0.5% xylan pH 6.0 The substrate was manufactured the day before the analysis: 1 g of beech xylan (xylan from beech wood, SERVA 38500) was dissolved at room temperature at magnetic in cohesion to 60 ml 5% NaOH. Add 100 ml 80 mM set-up buffer pH 6.0. The pH of the solution was adjusted to less than 6 (approx. 5.7-5.9) with concentrated hydrochloric acid and made up to 200 ml by water. The substrate solution was maintained overnight in the refrigerator, during which the pH may rise. The next day (on the day of use) the pH was adjusted to 6.0, if applicable, with 0.5 M NaOH or 0.5 M Hydrochloric acid.

### Hydrazide Reagent (0.5%)

### Stock Solution 5%

25 g of p-hydroxybenzoic acid hydrazide (PAHBAH; Alfa Aesar A12702,

LOT: 10190393 or similar) was dissolved in 0.5 M HCl, filled up to 500 ml, and filtered (e.g. Macherey-Nagel MN 616 ¼*Ø185 mm. Cat. No. 532 018). The stock solution was stored in fridge (2-8° C.) for a maximum of 6 weeks.

### Working Solution (0.5%)

2.325 g Titriplex III (EDTA, Merck 8418) was dissolved in approx. 200 ml of 0.5 M NaOH. 50 ml of stock solution was added and filled up to 500 ml of 0.5 M NaOH.

Alternatively, Xylanase activity measurement can be performed as described in U.S. Pat. No. 8,465,946 B2.

### EXAMPLE 2: SITE-DIRECTED MUTAGENESIS ON XYLANASES

Specific muteins of Bacillus subtilis xylanase were created by site directed mutagenesis of the wild type enzyme

AA exchanges
D11F
D11Y
S22E
G34D
R122D

### EXAMPLE 3: XYLANASE VARIANTS

### 3.1 Xylanase Production

For the B. subtilis expression strains, cultivations of the seed cultures were carried out at 37° C. and 180 rpm shaking in Erlenmeyer flasks with a maximum culture volume of 15% of the flask volume. The first pre-culture was prepared as overnight culture of one colony from a lysogeny broth (LB) agar plate (10 g/L bacto-tryptone, 5 g/L yeast extract, 10 g/L NaCl, 10 g/L agar) with suitable antibiotics. The secondary inoculum was cultured from 5% (v/v) of the primary inoculum. For the fermentations carried out, the secondary seed cultures were used as an exponentially growing culture to inoculate 0.5 L fermenters with an inoculum size of 3.75% (v/v). The composition of the media used was as described herein above. The culture temperature was 37° C., and the pH was 7.0+/−0.20 which was automatically adjusted with NH₃ and H₂SO₄ during the fermentation process of 65 h. The initial aeration and agitation rates were 0.5 vvm and 1200 rpm. Samples were taken over the entire fermentation process after a cultivation time of 16 h, 23.5 h, 40.0 h, 45.2 and 65.0 h. Feeding of glucose solution was started at cultivation point of pO₂<50% with a feeding rate of 2.5 g/h. After centrifugation for 15 min at 4500 rpm the culture supernatant was used for activity measurements and analysis via SDS Page. Differences to B. pumilus expression strain cultivation is a cultivation pH of 7.5+/−0.20, sucrose or glucose as carbon source and the glucose feeding of 3.0 g/h is started with an increased cultivation pH of 0.15.

### 3.2 Results

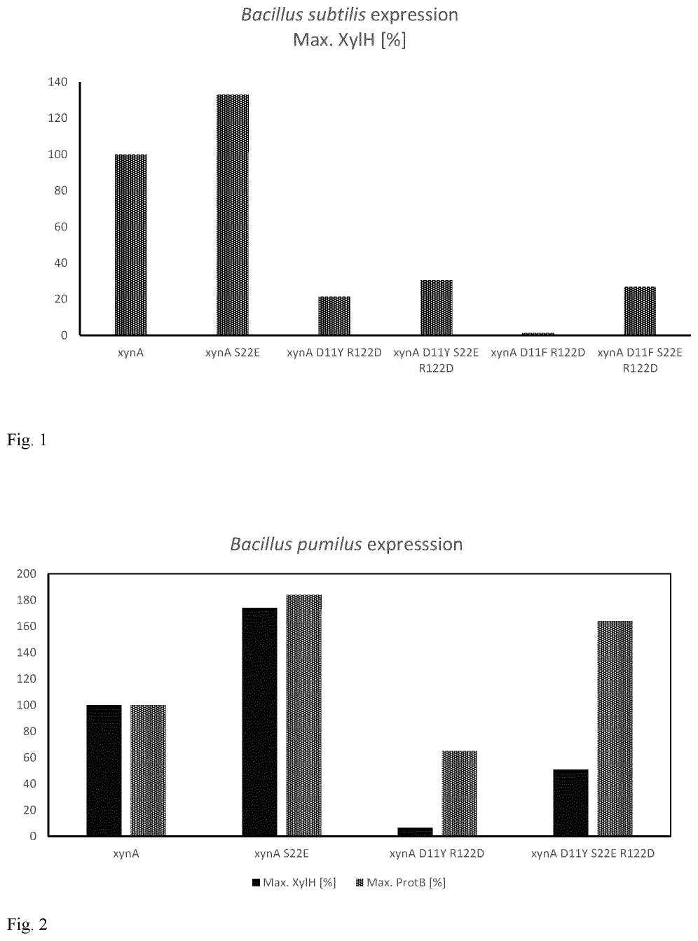

The xylanase variants containing amino acid exchange S22E in comparison to the parent enzyme were produced in Bacillus subtilis (FIG. 1 and Table 2).

TABLE 2
Relative maximal xylanase activity XylH of different xylanase
variants produced in Bacillus subtilis. Maximal xylanase
activity of the wildtype xylanase molecule was set as 100%.

| Xylanase variant XynA | Maximal relative xylanase |
|---|---|
| Modifications | activity XylH [%] |
| None (parent) | 100 |
| (SEQ ID NO: 1) | |
| S22E | 133 |
| D11Y R122D | 22 |
| D11Y S22E R122D | 31 |
| D11F R122D | 1.4 |
| D11F S22E R122D | 22 |

The xylanase variants containing amino acid exchange S22E in comparison to the parent enzyme produced in Bacillus pumilus shown by protein content in (g/L) and by xylanase activity (XylH) are shown in FIG. 2 and Table 3.

TABLE 3
Relative maximal xylanase activity (XylH) and amount (ProtB) of
the different xylanase variants produced in Bacillus pumilus.
Maximal xylanase activity and protein amount of the wildtype
xylanase molecule was set as 100%.

| | Maximal relative | |
|---|---|---|
| Xylanase variant | xylanase activity | Maximal protein |
| XynA Modifications | XylH [%] | amount ProtB [%] |
| None (parent) | 100 | 100 |
| (SEQ ID NO: 1) | | |
| S22E | 174 | 184 |
| D11Y R122D | 7 | 65 |
| D11Y S22E R122D | 51 | 164 |

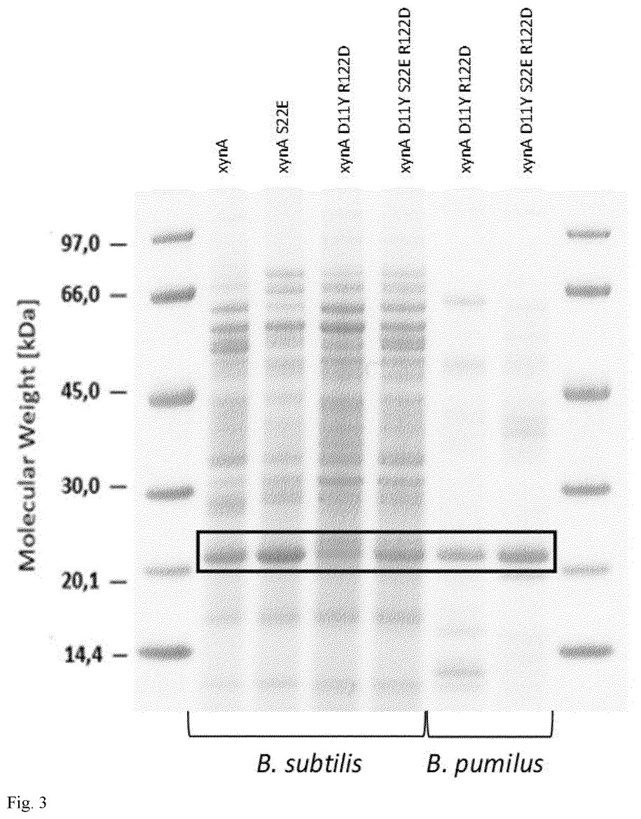

Results of production of xylanase variants containing amino acid exchange S22E in comparison to the parent enzyme and/or a variant without the S22E amino acid exchange in Bacillus subtilis and Bacillus pumilus by SDS Page are shown in FIG. 3.

The results show that introduction of amino acid exchange S22E results in increased xylanase volume yield compared to a xylanase polypeptide without the said amino acid exchange.

### EXAMPLE 4: BAKERY APPLICATIONS

### 3.1 General Methods and Methods for the Preparation and Evaluation of the Properties of the Dough and Bakery Products (Baking and Evaluation Process)

### General Procedure for Making the Breads

In the following a general outline of an exemplary procedure for making the breads is given.

1) Weighing all ingredients. Flours, salt, yeast, citric acid, gluten and other ingredients and the enzymes were added into a plastic bowl.

2) The ingredients were added to a spiral mixer Diosna SP12. The water temperature was adjusted to 34° C. and added into the mixer. All ingredients were mixed at 50 Hz for 3 minutes and 60 Hz for 7 minutes.

3) The dough was taken from the mixer with help of a plastic scraper and left to rest at room temperature for 10 minutes.

During resting, the dough temperature was measured using a standard thermometer (Testo 926 with PT100) to make sure that each dough has the same temperature (a variation between doughs of 1° C. is allowed). Dough temperature after mixing should be at 26-27° C.

The dough was manually evaluated (see below). Each dough was divided into exactly 500 gr pieces using scales and added into a bowl for fermentation. The dough was fermented at 32° C. and 80% relative humidity for 70 minutes.

4) Afterwards, the dough was baked at 240° C. for 32 minutes.

5) After the breads had cooled down, the volume was measured using a BVM 6630 (Perten Instruments). Final volume indicated was the average of at least three breads.

### Manual Dough and Bread Evaluation

Dough properties were evaluated directly after mixing, during the 20 minutes bench time and shaping. A scale of 1 to 10 indicates the dough properties, where dough 1 (reference) is given score 5 for all parameters and all other doughs are compared to dough 1. A higher number means that the described parameter is more or higher compared to the reference. A lower stickiness and softness are preferable and are represented by lower numbers. Properties of the bread were evaluated after baking.

TABLE 4
Bakery parameters

| Parameter | Definition | Scale |
|---|---|---|
| Stickiness | Degree to which the dough | Less sticky 0-4 |
| after mixing | sticks to the hands when | Same as control 5 |
| | dough is pressed by the | More sticky 6-10 |
| | palm of the hand, tested | |
| | directly after removing | |
| | dough from the mixer. | |
| Stickiness | Degree to which the dough | |
| after resting | sticks to the hands when | |
| | pressed by the palm of | |
| | the hand, tested 5 to 10 | |
| | minutes after removing | |
| | the dough from the mixer | |
| Extensibility | The ability to stretch a | Less extensible 0-4 |
| | dough by pulling a piece | Same as control 5 |
| | of dough on both ends | More extensible 6-10 |
| | without breaking | |
| Elasticity | The ability of a dough to | Less elastic 0-4 |
| | return to its original | Same as control 5 |
| | shape after being pulled | More elastic 6-10 |
| Softness | The amount of force needed | Less soft 0-4 |
| | to depress the surface of | Same as control 5 |
| | a dough piece | More soft 6-10 |
| Bread volume | Volume of a complete loaf | Measured in ml |
| | as measured using volume | |
| | scanner (Perten | |
| | Instruments) | |
| Bread shape | The height versus width | Just Round, Somewhat |
| | of a cross-section of | round, Round = |
| | bread | higher height vs. |
| | | width at equal volume |
| | | compared to control |
| | | Normal = No change |
| | | in height vs. width |
| | | at equal volume |
| | | compared to control |
| | | Just flat, Somewhat |
| | | Flat, Flat = more |
| | | width vs. height at |
| | | equal volume compared |
| | | to control |

### 3.2 Dough Properties, Bread Volume and Shape Using Different Xylanases

A comparison of xynA D11Y S22E R122D (xynA=SEQ ID NO: 1) against a commercially available xylanase enzyme product was performed. A bread was baked using the following recipe according to the described baking and evaluation process.

### Recipe

Wheat Flour: Grade 550, Germany

| | Amount |
|---|---|
| Ingredient | (w/w on total flour) |
| Water | 62% |
| Salt | 1.5% |
| Fresh yeast | 3% |
| Ascorbic acid | 50 ppm |

The enzyme doses are in ppm based on the weight of the flour. EP=enzyme protein.

TABLE 5
Dough and bread properties
100% Wheat flour white pan bread

| | | 2.5 XylH/kg flour xynA | 3 XylH/kg |
|---|---|---|---|
| | Control | D11Y S22E R122D | Powerbake 950 |
| Stickiness after | 5 | 3 | 5 |
| mixing | | | |
| Stickiness after | 5 | 3 | 5 |
| resting | | | |
| Extensibility | 5 | 5 | 6 |
| Elasticity | 5 | 5 | 5 |
| Softness | 5 | 4 | 4 |
| Bread volume | 1624 | 1730 | 1699 |
| [ml] | | | |

The results in Table 5 show that an optimal dosage of 2.5 XylH/kg gives decreased dough stickiness and a firmer dough than a control dough without xylanase and a reference dough with a commercially available xylanase benchmark.

3.3 Comparison of xynA D11Y S22E R122D to xynA D11Y R122D in Baking

A bread was baked using the following recipe and process:

### Recipe

Wheat Flour: Grade 550, Germany

| | Amount |
|---|---|
| Ingredient | (w/w on total flour) |
| Water | 62% |
| Salt | 1.5% |
| Fresh yeast | 3% |
| Ascorbic acid | 50 ppm |

A bread was prepared using xynA D11Y S22E R122D xylanase in comparison to xynA D11Y R122D. The enzyme doses and the results are given in the following Table 6.

TABLE 6
Dough and bread properties
100% wheat flour, white pan bread

| | Reference | 5 XylH/kg xynA | 5 XylH/kg xynA |
|---|---|---|---|
| | No enzyme | D11Y R122D | D11Y S22E R122D |
| Stickiness | 5 | 4 | 3 |
| after mixing | | | |
| Stickiness | 5 | 4 | 3 |
| after resting | | | |
| Extensibility | 5 | 6 | 5 |
| Elasticity | 5 | 5 | 5 |
| Softness | 5 | 4 | 3 |
| Bread | 1620 | 1811 | 1870 |
| volume [ml] | | | |

The results show that an optimal dosage of 5 XylH/kg flour of xynA D11 Y S22E R122D gives decreased stickiness of the dough after mixing and after around 10 minutes resting time, compared to the xynA D11Y R122D and reference dough. The resulting bread volume is significantly higher.

### REFERENCES

1. Agaisse and Lereclus, 1994, Molecular Microbiology 13:97
2. Alptoni et al. (2016), Int J Biol Macromol 87:522
3. Courtin et al. (1999) J Agric Food Chem. 47(5):180-7
4. DeBoer et al., 1983, Proc. Natl. Acad. Sci. USA 80:21
5. Egon et al., 1988, Gene 69:301
6. EP 2 145 006 B1
7. Hue et al., 1995, Journal of Bacteriology 177:3465
8. Ruller et al. (2014) Protein Engineering, Design & Selection 27(8):255
9. Simonen and Palva, 1993, Microbiological Reviews 57:109
10. Villa-Kamaroff et al., 1978, Proc. Natl. Acad. Sci. USA 75:3727
11. WO 94/25612
12. WO 99/43835
13. WO 01/066711 A1
14. WO 2010/072225 A1

1. A xylanase polypeptide comprising
(i) an amino acid sequence at least 70% identical to SEQ ID NO:1,
(ii) an amino acid exchange at the position corresponding to amino acid D11 in SEQ ID NO:1; and
(iii) an amino acid exchange at the position corresponding to amino acid S22 in SEQ ID NO:1.

2. The xylanase polypeptide of claim 1, wherein said amino acid exchange at the position corresponding to amino acid D11 in SEQ ID NO:1 is a D11Y or a D11F exchange.

3. The xylanase polypeptide of claim 1, wherein said amino acid exchange at the position corresponding to amino acid S22 in SEQ ID NO:1 is an S22E exchange.

4. The xylanase polypeptide claim 1, wherein said xylanase polypeptide further comprises an amino acid exchange at the position corresponding to amino acid R122 in SEQ ID NO:1.

5. The xylanase polypeptide of claim 1, wherein said amino acid exchange at the position corresponding to amino acid R122 in SEQ ID NO:1 is an R122D or R122N exchange.

6. The xylanase polypeptide of claim 1, wherein said xylanase polypeptide comprises, preferably consists of, the amino acid sequence of any one of SEQ ID NOs:3 to 6 and 10 to 13, preferably of SEQ ID NOs:3 to 6, more preferably of SEQ ID NO:5.

7. A polynucleotide comprising a nucleic acid sequence encoding a xylanase polypeptide according to claim 1.

8. The polynucleotide of claim 7, wherein said polynucleotide is comprised in an expression construct.

9. A host cell comprising the xylanase polypeptide according to claim 1.

10. The host cell of claim 9, wherein said host cell is a bacterial cell of the genus Bacillus, preferably a B. pumilus or a B. subtilis cell.

11. A method of producing a xylanase polypeptide comprising expressing a polynucleotide according to claim 7 in a host cell.

12. A method of producing a bakery product comprising
(a) admixing a xylanase polypeptide according to claim 1 to flour and water, and
(b) incubating the admixture of step (a) for an incubation period.

13. The method of claim 12, wherein said bakery product is a dough or a baked good.

14. A composition comprising a xylanase polypeptide according to claim 1 and at least one additive and/or at least one flour.

15. (canceled)

16. The Method of claim 11, wherein said host cell is a bacterial cell of the genus Bacillus, preferably a B. pumilus or a B. subtilis cell.
